(19) United States
(12) Patent Application Publication
(10) Pub. No.: US 2010/0034879 A1
(43) Pub. Date: Feb. 11, 2010

(54) HYALURONIC ACID PRODUCT AND METHOD FOR TREATING LACERATIONS AND WOUNDS IN A LIVING BODY

(76) Inventor: Hilton Becker, Boca Raton, FL (US)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/189,536

(22) Filed: Aug. 11, 2008

(51) Int. Cl. A61K 9/113; A61K 31/728

(52) U.S. Cl. 424/484; 514/54

(57) ABSTRACT

A method for texturing the surface of a breast implant includes the step of partially impregnating a silicone outer surface of the implant with particles of a biologically active material such as acellular dermis of human or animal origin and/or hyaluronic acid granules. The biologically active material promotes tissue ingrowth into a plurality of cavities filled with a biologically active material.

### FIELD OF THE INVENTION

This invention relates to a hyaluronic acid product and method for treating lacerations and wounds in a living body and more particularly to a hyaluronic acid gel and method for applying hyaluronic acid gel to wounds or abraded skin in a preservative free form.

### BACKGROUND FOR THE INVENTION

It is known that hyaluronic acid (HA) provides a favorable environment for cell growth and motility that may foster tissue regeneration instead of fibrosis. This observation may have therapeutic implications in that HA applications may modulate healing of adult wounds in a manner more similar to the manner of fetal wound healing. These facts and observations were reported in an article entitled “Using Hyaluronic Acid to Create a Fetal-like Environment In Vitro by Scott Shepard, Hilton Becker and James Hartmann that was published in the Annals of Plastic Surgery, Volume 36, No. 1 January 1996. The article went on to state: “Our study shows that the closure of a mechanical disruption (in vitro wound) of this type is accelerated by the addition of exogenous HA in the presence of adult serum . . . .” These findings suggest that large amounts of HA enhance the proliferation of fibroblasts from dermal tissue and promote the phenotypic changes associated with the migration of fibroblasts into a wound.

Hyaluronic acid gel is a naturally occurring disaachoride found in the skin and joints. It has been shown to have a stimulatory effect on cell division thus accelerating healing. HA is water solvable. However, once dissolved in water, due to its high nutritive value, it is rapidly prone to bacterial contamination and ingrowth. Therefore, preservatives must be added to prevent bacterial growth. This growth inhibiting effect is undesirable for the healing of wounds or abraded skin.

Squalane oil is a botanical lipid that duplicates the molecular structure and weight of human lipids. Scientists have found that the skin's sebaceous glands synthesize approximately 10-12% Squalane. However, today most Squalane oil is derived from plants as opposed to the animal derived (shark liver oil). It has been found that the valuable lipid produced from olives is extremely compatible with the skin and safe. It has also been found that Squalane oil permeates into the skin at a rate of 2 mm/second.

Increasing use of hyaluronic acid in cosmetic and other surgery has lead to an increase in the use of hyaluronic acid gel to speed up healing and reduce scarring. Accordingly, it is presently believed that there is a need for a relatively pure hyaluronic acid that is free of preservatives and at the same time free of harmful bacteria. It is also believed that there is a relatively large market for any such product. It is also believed that the hyaluronic products in accordance with the present invention are free of preservatives and at the same time enhance healing of lacerations and wounds in the human body.

### BRIEF SUMMARY OF THE INVENTION

In essence, the present invention contemplates a water activatable medicinal preparation for treating lacerations and wounds in a human body. The medicinal preparation is an emulsion of finely ground hyaluronic acid dispersed in a human compatible oil and preferably in a human compatible absorbable oil such as Squalane oil. In a preferred embodiment of the invention the emulsion contains about 5% to about 20% hyaluronic acid by weight or by volume. In a more preferred embodiment of the invention the particles size of the hyaluronic acid is equal to or less than about 20 microns.

The present invention also contemplates a method for treating lacerations and wounds in a living body such as a human being in order to enhance healing and reduce scarring. The method includes the steps of providing a water activatable emulsion of relatively pure hyaluronic acid in a human compatible and preferably highly absorbable oil. The hyaluronic acid is preferably ground to a fine particle size of about 20 microns or less and mixed into a dispersion in the human compatible absorbable oil to produce an emulsion with about 5% to about 20% by weight or volume hyaluronic acid. The emulsion is then applied to the laceration or wound by any means as will be well understood by a doctor or surgeon of ordinary skill in the art and activated with water to form hyaluronic acid gel. Typically the application of water may be by a fine spray but, other methods may be used. The oil is absorbed by the body while a portion of the water additives remain in contact with the hyaluronic acid and forms a hyaluronic acid gel which enhances the healing of the laceration and/or wound.

The invention will now be described in connection with the accompanying figures.

### DESCRIPTION OF THE DRAWINGS

FIG. 1 is a flow chart illustrating the formation of a water activated emulsion for treating human injuries namely lacerations and wounds and a method for treating lacerations and wounds with a water activatable emulsion;

FIG. 2 is a photograph of an open wound following surgery;

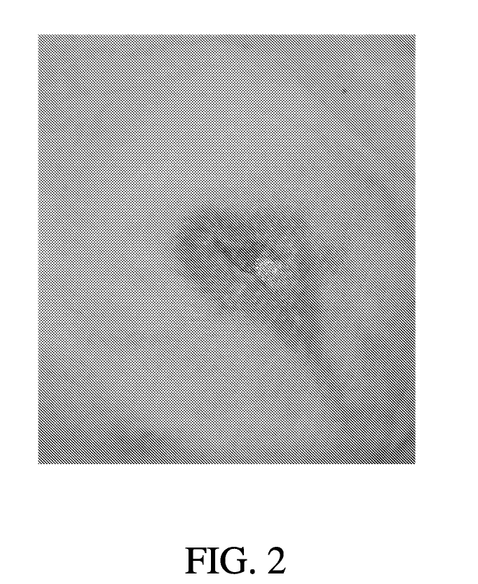

FIG. 3 is a photograph of the wound shown in FIG. 2 with the application of Hyaluronic Acid/Oil;

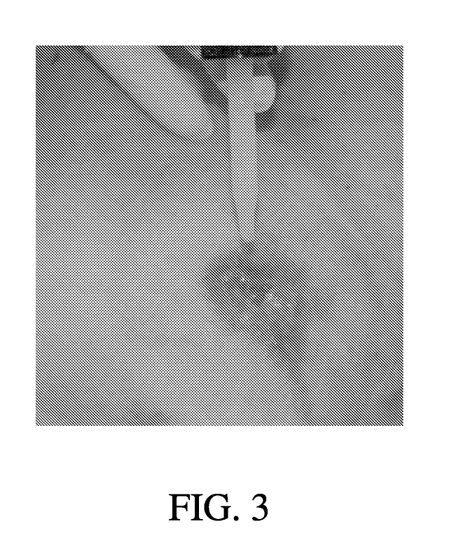

FIG. 4 is a photograph of the wound shown in FIGS. 2 and 3 after spraying with water to form a gel;

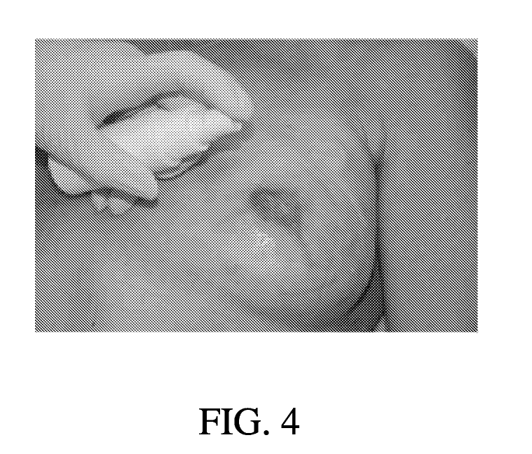

FIG. 5 is a photograph of the wound shown in FIGS. 2-4 but covered with the gel formed in FIG. 4; and

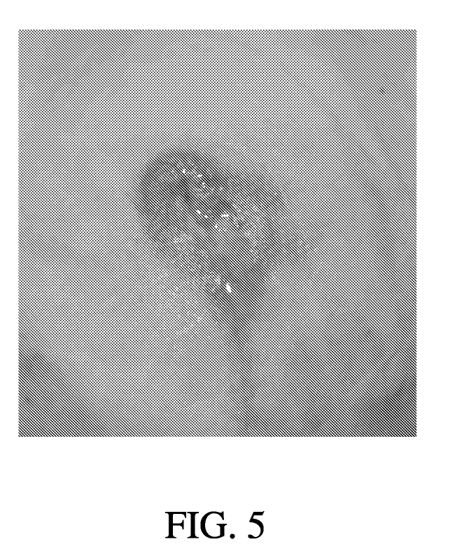

FIG. 6 is a photograph of the wound shown in FIGS. 2-5 after healing for 10 days.

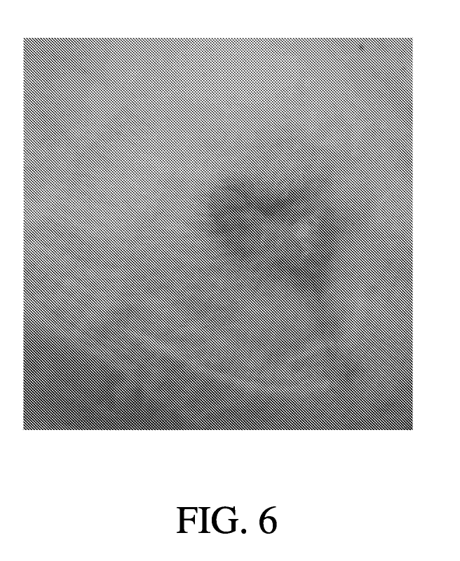

### DESCRIPTION OF THE PREFERRED EMBODIMENTS OF THE INVENTION

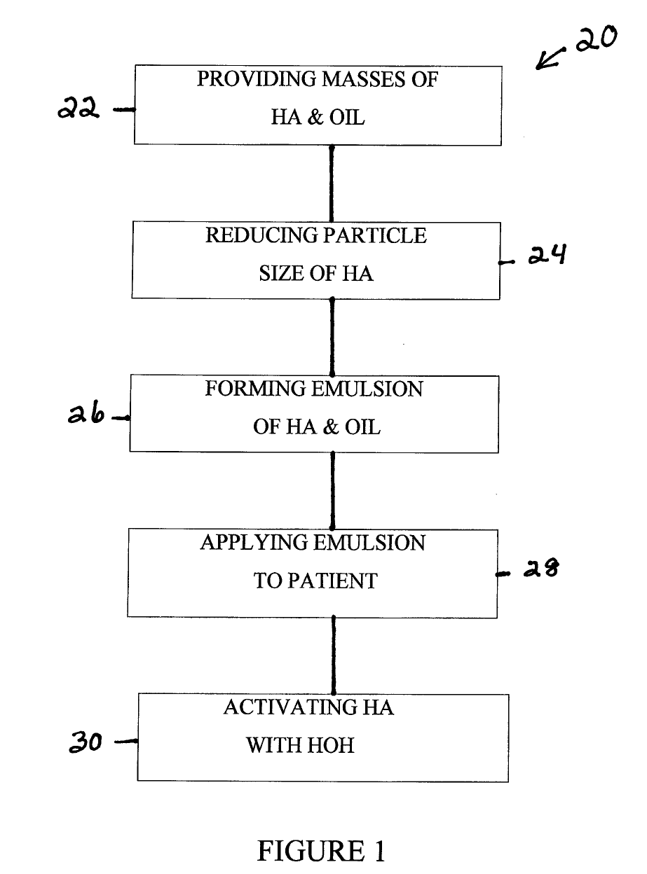

FIG. 1 illustrates a first embodiment of the invention i.e. a method for treating lacerations or wounds in a living body. The method includes the step 20 of providing a first mass of a preservative free hyaluronic acid in relatively pure form and a second mass of a human compatible and prefereably absorbable oil as for example Squalane oil.

Hyaluronic acid is a glycosaminoglycan (GAG) and is present in cartilage as a coating around each cell and is also found in skin and other tissues. Hyaluronic acid helps wounds to heal more quickly and can reduce the appearance of scars. Medical grade hyaluronic acid is available in average particle sizes ranging from about 20 to about 700 microns.

In the practice of the present invention, it is believed that the smaller the particle size of the hyaluronic acid, the better the result. Accordingly, it is presently believed that the 20 micron particles size can be further reduced by grinding. Samples of larger particles were also reduced by grinding or on a small scale with a mortar and pestle. Thus, the second step 24 in the present invention includes reducing the hyaluronic acid particles to about 20 microns and preferably to about 20 microns or less.

Squalane oil is a botanical lipid derived from olives, has a molecular formula of C₃₀H₆₂ and permeates into the skin of a human at a rate of 2 mm/sec. Squalane oil is human compatible, rapidly absorbed into the body and because of the deep penetration assists in the acceleration of cell growth. It is believed that other human compatible oils may be used in the practice of the invention, however absorbable oils and preferably highly absorbable oils are preferred.

In step 26, the finely ground hyaluronic acid is thoroughly mixed with the human compatible highly absorbable oil to form an emulsion i.e. a mixture of two immiscible (unblendable) substances. One substance (the dispersed phase) is dispersed in the other (the continuous phase). The resulting mix may also be defined as a dispersion wherein the dispersed phase is suspended in a dispersion medium. The mixing maybe by conventional means. The resulting mix can also be defined as a suspension wherein microscopically visible particles are dispersed throughout a less dense liquid where they are easily filtered out, but not easily settled.

The emulsion of hyaluronic acid particles in a human compatible absorbable oil is applied to a wound or laceration in step 28. This application is done in a conventional manner as will be well understood by persons of ordinary skill in the art such a surgeons and more particularly cosmetic surgeons.

In step 30 a cosmetic surgeon applies water to the emulsion as for example by a fine spray or other means. It is presently believed that the absorbable oil rapidly penetrates the skin leaving a portion of the hyaluronic acid particles on the surface and that the water reacts with the hyaluronic acid particle to form a hyaluronic acid gel at the wound site or on the laceration as well as into the dermis. It is also believed that a portion of the hyaluronic acid gel is carried into the patient's dermis while a portion covers the wound area to enhance healing.

A further advantage of a highly absorbable oil such as Squalane oil is that it leaves little or no oily residue at the surface.

A hyaluronic acid product in accordance with the present invention comprises an emulsion, dispersion or suspension of relatively fine preservative free hyaluronic acid particles of less than about 70 microns and preferably less than about 20 microns in a rapidly absorbable human compatible oil. The product is prepared as discussed above and is activatible by the application of water to a coating or thin film of the product on a patient's wound. In the preferred embodiment of the invention the emulsion contains between 5-20% hyaluronic acid and 95% to about 80% of Squalane oil wherein the percentage given is percent by weight and/or approximately the same percentage by volume.

While the invention has been described in connection with its preferred embodiments it should be recognized that changes and modifications may be made therein without departing from the scope of the appended claims.

What is claimed is:

1. A method for treating lacerations and wounds in a living human body comprising the steps of:
providing a first mass of a preservative free hyaluronic acid and a second mass of a human compatible oil;
reducing the particle size of the hyaluronic acid to produce ultra-fine particles of hyaluronic acid;
forming an emulsion of the fine particles in the human compatible oil;
applying the emulsion to the laceration or wound of the patient; and
forming an hyaluronic acid gel by applying water to the emulsion on the laceration or wound of the patient.

2. A method for treating lacerations and wounds in a human body according to claim 1 in which the human compatible oil is rapidly absorbable into the skin of a patient.

3. A method for treating lacerations and wounds in a human body according to claim 2 in which the human compatible highly absorbable oil is squalane oil.

4. A method for treating lacerations and wounds in a human body according to claim 3 in which the hyaluronic acid consists of pure hyaluronic acid.

5. A method for treating lacerations and wounds in a human body according to claim 4 in which the hyaluronic acid particles have a particle size of less than about 70 microns.

6. A method for treating lacerations and wounds in a human body according to claim 4 in which the hyaluronic acid particles have a particle size of about 20 microns or less.

7. A method for treating lacerations and wounds in a human body according to claim 6 in which the hyaluronic acid gel is formed by spraying water onto the emulsion.

8. A method for treating lacerations and wounds in a human body according to claim 7 in which the emulsion contains between about 5% and about 20% by weight hyaluronic acid and in which between 95% and 80% by weight squalane oil.

9. A hyaluronic acid product for treating lacerations and wounds in a living body comprising an emulsion of hyaluronic acid in a human compatible highly absorbable oil.

10. A hyaluronic acid product for treating laceration and wounds in a living body according to claim 9 in which the human compatible highly absorbable oil is squalane oil.

11. A hyaluronic acid product for treating laceration and wounds in a living body according to claim 10 wherein the preparation contains about 5 to about 20% by weight hyaluronic acid and about 95% to about 80% by weight squalane oil.

12. A hyaluronic acid product for treating laceration and wounds in a living body according to claim 11 in which the hyaluronic acid in the emulsion has a particle size of less than about 70 microns.

13. A hyaluronic acid product for treating laceration and wounds in a human body according to claim 12 in which the hyaluronic acid in the emulsion has a particle size of 20 microns or less.

14. A method for preparing a hyaluronic acid product according to claim 14 in which the particle size of the hyaluronic acid is reduced to about 20 microns or less.

15. A method for preparing a hyaluronic acid product according to claim 15 wherein the product contains from 5-20% by weight hyaluronic acid and between about 95-80% by weight squalane oil.
